SEQUENCE LISTING (8 sequences)

SEQ ID NO: 1 - DNA, 21 bp, Artificial primer
  Target is rs7576247
ttcagaacaccctagcagatg

SEQ ID NO: 2 - DNA, 20 bp, Artificial primer
  Target is exon 6(A>G)
ttgcaaaacctgcataacaa

SEQ ID NO: 3 - DNA, 21 bp, Artificial primer
  Target is rs11553742
aggccaacctgaactcctctg

SEQ ID NO: 4 - DNA, 20 bp, Artificial primer
  Target is exon 3(C>T)
cctgtcttgctttatgggct

SEQ ID NO: 5 - DNA, 10 bp, Homo sapiens
aatacttttg

SEQ ID NO: 6 - DNA, 282 bp, Homo sapiens
acatttaaggcttagactcataaataatgctattgtttatgatttgaaaactttcaggcaaaatccaatttacatttttcccttccctagcaattacttttttccagcttcaactcttcttagttactaatactttgttgactttaaaaatgaaatcattcacaaacttttggtatatgatggagaatgaaaaactagagtcagacagctttaattgacattgtcaacacctccagttatcaggaatacatttttttactgccttaacctgtagtgcgtaga

SEQ ID NO: 7 - DNA, 18 bp, Artificial primer
  Target is rs2438211
acatttaaggcttagact

SEQ ID NO: 8 - DNA, 20 bp, Artificial primer
  Target is (C>T)
tctacgcactacaggttaag

(19) United States
(12) Patent Application Publication
(10) Pub. No.: US 2015/0259750 A1
(43) Pub. Date: Sep. 17, 2015

(54) GENE-GENE MODEL FOR ESTIMATING CANCER SUSCEPTIBILITY, RISK OF MULTIPLE TUMOR SITES, AND THE AGGRESSIVENESS OF PROSTATE CANCER

(71) Applicant: Radhika Gade Andavolu, Rancho Mirage, CA (US)

(72) Inventor: Radhika Gade Andavolu, Rancho Mirage, CA (US)

(73) Assignee: GENETIC RESEARCH INSTITUTE OF THE DESERT, Rancho Mirage, CA (US)

(21) Appl. No.: 14/554,946

(22) Filed: Nov. 26, 2014

Related U.S. Application Data
(60) Provisional application No. 61/963,206, filed on Nov. 26, 2013.

(51) Int. Cl. C12Q 1/68

(52) U.S. Cl. CPC C12Q 1/6886; C12Q 2600/156; C12Q 2600/112; C12Q 2600/172

(57) ABSTRACT

The present disclosure provides methods for estimating cancer susceptibility, risk of developing multiple tumor sites, and the aggressiveness of prostate cancer. Disclosed are interactions among three polymorphisms: the Acid Phosphatase Locus-1 (ACP1) 228C>T (rs11553742) and 413A>G (Q106R, rs79716074) single nucleotide polymorphisms (SNPs), and a SNP for the Astrocyte Elevated Gene-1 (AEG1) (rs2438211). Additionally interactions among these polymorphisms are disclosed to modify the underlying quantitative expression of major cancer-related genes, indicating that optimization of these gene products by pharmacologic or gene therapy interventions would be beneficial as a cancer therapy to reduce the risk of tumor metastasis, and would also be beneficial in preventing cancer.

### CROSS-REFERENCE TO RELATED APPLICATIONS

This application claims the benefit of U.S. Provisional Patent Application No. 61/963,206, filed Nov. 26, 2013, the entire contents of which are hereby incorporated by reference.

### FIELD OF THE DISCLOSURE

The present disclosure relates to genetic methods of estimating cancer susceptibility, risk among cancer cases of developing multiple tumor sites, and risk among prostate cancer cases of having a more aggressive tumor type requiring either prostatectomy or orchiectomy.

### BACKGROUND OF THE DISCLOSURE

Genetic variants predicting cancer susceptibility are important primarily because they identify molecules and pathways involved in tumorigenesis that may have implications for pharmaceutical development. However, most variants are likely to have only marginal pre-diagnostic relevance and, excepting the rare mutations of BRCA1, BRCA2 and HER2, they have limited post-diagnostic impact on patient care. The greater need in cancer genetics is the identification of variants that distinguish slow-growing tumors from more fast-growing lethal tumors, both to facilitate novel drug therapies and to signal the need for more aggressive treatment regimens.

A recent review of genetic risk prediction in complex diseases concluded that only a few molecular genetic variables have been found to play an important role in historical risk predictions: BRCA1 and BRCA2 (Jostins, L., and Barrett, J. C., Hum. Mol. Genet. 20(R2):R182-8 (2011)). This view is widely held despite large scale studies showing that among incident breast cancer cases only 2.9% carried BRCA1 mutations and 2.2% had BRCA2 mutations (Goodwin, P. J., et al., J. Clin. Oncol. 30(1):19-26 (2012)). Similarly, results from the NCI Breast and Prostate Consortium (Lindström, S., et al. Cancer Epidemiol. Biomarkers Prev. 21(3):437-44 (2012)) revealed that the cumulative percent of the variance in prostate cancer risk accounted for by the additive power of 25 of the most common prostate cancer genetic markers was only 4%. These studies serve to underscore the need for a shift away from additive gene-gene model towards a more comprehensive multiplicative model of disease risk profiles.

Single nucleotide polymorphisms (SNPs) are defined as genetic variation in a DNA sequence of a particular gene. SNPs are usually considered to be point mutations can occur in coded or non-coded regions. Point mutations have been evolutionarily successful enough to recur in a significant proportion of the population and these variants may or may not affect the function of the gene.

AEG-1/MTDH/LYRIC was initially identified as a HIV-1-inducible gene in primary human fetal astrocytes (Kang, D. C., et al., Gene 353(1):8-15 (2005); and Su, Z. Z., et al., Oncogene 21(22):3592-602 (2002)). Astrocyte-elevated gene-1 (AEG-1) over expressed in human brain and prostate cancers promotes angiogenesis and metastasis (Emdad, L., et al., Pharmacol. Ther. 114(2):155-70 (2007), erratum in Pharmacol. Ther. 115(1):176 (2007); Hu, G., et al., Clin Cancer Res. 15(18):5615-20 (2009); and Song, L., et al., J. Pathol. 219(3):317-26 (2009)). Astrocyte Elevated Gene-1 (AEG-1) is ubiquitously overexpressed in all or most cancers and plays a regulatory role in diverse and multiple processes of carcinogenesis. AEG1 is a multi-functional regulator of normal and abnormal physiology; it contributes to broad-spectrum resistance to various chemotherapeutics; and it was recently proposed as the first potential ‘pan-cancer’ gene (Byoung, K. Y., et al., Pharmacol. Ther. 130(1):1-8 (2011)). However, despite its critical relevance to carcinogenesis, until now genetic polymorphisms for this important gene have not been studied for association with cancer susceptibility. For example, though studies have reported AEG-1 gene expression in animal models and tumor tissues, no studies have been reported in Caucasians on the AEG-1 gene polymorphisms in large samples to establish an association with cancers. A study from China screening variants in the AEG-1 gene detected 3 SNPs rs2438211, rs2449512, and rs1311 in linkage, but showed lack of correlation of the variants to disease progression in breast cancer (Liu, X., et al., PLoS One 6(3):e17582 (2011)). However, no studies have been reported screening AEG-1 gene polymorphisms in melanoma and prostate cancer.

Acid phosphatase locus-1 (ACP1) regulates the phosphorylation status of a number of proliferative signaling molecules and is over expressed in high potential metastasis tumor cells (Malentacchi, F., et al. Biochem. Biophys. Res. Commun. 334:875-883 (2005); and Song, D. X., et al., Zhonghua Yi Xue Za Zhi 88(17):1197-201 (2008)). Recently, it was found that ACP1 is highly connected with migration and invasion activities of carcinomas, where it is upregulated (Alho, I., et al., Tumour Biol. 34(4):1979-89 (2013); and Alho, I., et al., Cancer Genet Cytogenet. 181(1):20-4 (2008)). Though these few studies have shown the relevance of ACP to carcinogenesis there have been no large scale studies of ACP1 as a susceptibility marker for cancer.

### SUMMARY OF THE DISCLOSURE

Disclosed are genotyping polymorphisms for the ACP1 gene (rs11553742 and rs79716074) creating an ACP1 haplotype that provides an accurate and reliable estimate of risk for breast cancer.

Disclosed are genotyping polymorphisms for the ACP1 gene (rs11553742 and rs79716074) creating an ACP1 haplotype that provides an accurate and reliable estimate of risk for malignant melanoma.

Disclosed are genotyping polymorphisms for the ACP1 gene (rs11553742 and rs79716074) creating an ACP1 haplotype that provides an accurate and reliable estimate of risk for prostate cancer.

Disclosed are genotyping polymorphisms for the AEG1 gene (rs2438211) providing an accurate and reliable estimate of risk for prostate cancer.

Disclosed are genotyping polymorphisms for the AEG1 gene (rs2438211) providing an accurate and reliable estimate of risk for malignant melanoma.

Disclosed are genotyping polymorphisms for the AEG1 gene (rs2438211) and ACP1 haplotype (rs11553742 and rs79716074) creating a gene-gene combination that provides an accurate and reliable estimate of risk for prostate cancer.

Disclosed are genotyping polymorphisms for the AEG1 gene (rs2438211) and ACP1 haplotype (rs11553742 and rs79716074) creating a gene-gene combination that provides an accurate and reliable estimate of risk for breast cancer.

Disclosed are genotyping polymorphisms for the AEG1 gene (rs2438211) and ACP1 haplotype (rs11553742 and rs79716074) creating a gene-gene combination that provides an accurate and reliable estimate of risk for melanoma.

Disclosed are genotyping polymorphisms for the AEG1 gene (rs2438211) and ACP1 haplotype (rs11553742 and rs79716074) creating a gene-gene combination that provides an accurate and reliable estimate among cancer patients for risk of developing multiple tumors.

Disclosed are genotyping polymorphisms for the AEG1 gene (rs2438211) and ACP1 haplotype (rs11553742 and rs79716074) creating a gene-gene combination that provides an accurate and reliable estimate among prostate cancer cases of risk of developing more aggressive forms of prostate cancer.

Disclosed are modifications of the biological substrates of ACP1 or AEG1 or both that could be used prophylactically to reduce cancer risk.

Disclosed are modifications of the biological substrates of ACP1 or AEG1 or both that could be used among individuals diagnosed with cancer to reduce the risk of developing multiple tumor sites.

Disclosed are modifications of the biological substrates of ACP1 or AEG1 or both that could be used among individuals diagnosed with prostate cancer to reduce the risk of more aggressive cancer progression.

### DETAILED DESCRIPTION OF THE DISCLOSURE

### AEG1 and ACP1 Polymorphisms as Risk Markers for Cancer and Multiple Tumors

Astrocyte Elevated Gene-1 (AEG1) has recently been described as the first potential “pan-cancer” gene. However, until now polymorphisms for the AEG1 have not been studied. Similarly, several studies have shown that the gene for Acid Phosphatase Locus-1 (ACP1) plays a role in tumorigenesis, but it too has not been studied in large samples.

Studied were AEG1/ACP1 in samples of 758 samples comprised of 366 healthy controls as well as 199 breast cancer cases, 114 prostate cancer cases and 109 cases of malignant melanoma. All analyses were age- and sex-matched comparisons of non-Hispanic Caucasian subjects. Disclosed is the detection of AEG1 gene polymorphisms and the construction of gene-gene risk alleles in combination with ACP1 haplotypes to assess the risk of cancer occurrence test that is simple, reliable, faster and cost effective.
1. Individually, both AEG1 and ACP1 were robustly associated with cancer occurrence. Moreover, the gene-gene combination predicted the occurrence of melanoma (X2=76.611 p<0.00000), prostate cancer (X2=22.146 p<0.00000), and breast cancer (X2=10.687 p<0.001)
2. AEG1/ACP1 high-risk alleles were in turn associated with having multiple tumor sites (p=0.015).
3. AEG1/ACP1 risk alleles can be used as a screening device for cancer susceptibility to inform the need for more intensive ongoing cancer surveillance.
4. AEG1/ACP1 risk alleles can be used in newly diagnosed cancers to estimate the risk of acquiring multiple tumors (typically lethal) to inform the need for more intensive therapies.
5. Given the unprecedented role of AEG1/ACP1 in tumorigenesis demonstrated by our findings, modification of AEG1/ACP1 substrates may be useful as either a prophylactic treatment to reduce cancer risk or as a pharmacotherapy for the treatment of existing cancers.

TABLE 1
FREQUENCY DISTRIBUTION OF RISK ALLELES IN
CANCERS AND CONTROL GROUPS
AEG1/ACP1 RISK ALLELES

| SAMPLE | 1 | 2 | 3 | 4 | 5 | TOTAL |
|---|---|---|---|---|---|---|
| CONTROL (N) | 229 | 99 | 24 | 13 | 1 | 366 |
| | 62.6 | 27 | 6.6 | 3.6 | 0.3 | 48.3 |
| | 59.2 | 42.7 | 32.4 | 23.6 | 10 | |
| CANCER (N) | 158 | 133 | 50 | 42 | 9 | 392 |
| | 40.3 | 33.9 | 12.8 | 10.7 | 2.3 | 51.7 |
| | 40.8 | 57.3 | 67.6 | 76.4 | 90 | |

AEG1/ACP1 were studied in 758 samples. Since one individual had two cancers and there were 30 individuals (samples) with more than one cancer, the total shows 788 samples; in fact the sample size is only 758. The sample population was split to estimate the risk based on types of cancer and hence 30 cases more. To estimate the risk of cancer which the above table shows has only the actual individuals studied 758 with no sample duplication. All analyses were age- and sex-matched comparisons of non-Hispanic Caucasian subjects. DNA was isolated from all cancer patients and controls using standard procedure. An informed signed consent was obtained from all the study subjects.

Here, three genetic polymorphisms (rs7576247; rs11553742) in ACP gene and (rs2438211) in AEG-1 in cancer patients and healthy controls were screened to determine whether:
i) a particular SNP has significant influence on the susceptibility of cancer or to predict progression of disease; and
ii) if combination of SNPs from two different genes had more influence on the susceptibility of cancer or predict progression of disease.

### ACP1 Gene Polymorphisms

A PCR-RFLP method was used to screen ACP1 gene polymorphisms. ACP1 alleles (i.e., ACP1*A, ACP1*B, and ACP1*C) were determined with 2 of the selected SNPs: rs11553742 (C>T) is a synonymous polymorphism located in the codon 44 (exon 3) and rs7576247 (A/G) encodes an amino acid change in the codon 105 (exon 6) from arginine, present in ACP1*A allele, to glutamine in ACP1*B and *C alleles (Lazaruk, K. D., et al., Biochem. Biophys. Res. Commun. 15; 196(1):440-6 (1993)). Genotype and haplotype analysis were performed using PASW 18 (SPSS IBM).

| | Primer | Size | Cut | Restriction |
|---|---|---|---|---|
| Target | sequence 5′-3′ | (bp) | size | Enzyme |
| rs7576247 | For: 5′-TTCAGAAC | 299 bp | 199, | 2U TaqI |
| Exon 6 | ACCCTAGCAGATG-3′ | | 100 | @ 65° |
| (A > G) | Rev: 5′-TTGCAAAA | | | for 6 |
| | CCTGCATAACAA-3′ | | | hours |
| rs11553742 | For: 5′-AGGCCAAC | 341 bp | 255, | 2U HhaI |
| Exon 3 | CTGAACTCCTCTG-3′ | | 86 | @ 37° |
| (C > T) | Rev: 5′-CCTGTCTT | | | Overnight |
| | GCTTTATGGGCT-3′ | | | |

### AEG-1 Gene Polymorphism

Disclosed is a method of screening AEG1 using PCR-RFLP. The methods of Liu et al., the contents of which are hereby incorporated by reference in their entirety, were used to sequence the AEG-1 gene. This is the first study design of PCR-RFLP method screening rs2438211 (3′UTR-C/T) variant. Below a normal sequence is shown and no enzyme is found to recognize the site. A ‘T’ was inserted in the sequence replacing ‘C’ in the sequence and the search found SspI restriction enzyme recognition mutation site ‘T’ in sequence (AATAC/TTTTG) where it cuts the PCR product in individual carrying this mutant ‘T’ (named 2 allele) while the wild type ‘C’ (1-allele) remains uncut. This method is simple, faster and cost effective.

rs2438211 Normal Sequence:

acatttaaggcttagactCATAAATAATGCTATTGTTTATGATTTGAAAACTTTCAGGC
AAAATCCAATTTACATTTTTCCCTTCCCTAGCAATTACTTTTTTCCAGCTTCAACTCTTCTTAG
TTACTAATAcTTTGTTGACTTTAAAAATGAAATCATTCACAAACTTTTGGTATATGATGGAGAA
TGAAAAACTAGAGTCAGACAGCTTTAATTGACATTGTCAACACCTCCAGTTATCAGGAATACAT
TTTTTTACTGCcttaacctgtagtgcgtaga (225 bp)

| | Primer | Restriction |
|---|---|---|
| Target | sequence 5′→3′ | Enzyme |
| rs2438211 | AEGF: 5'- ACA TTT AAG | SspI |
| (C > T) | GCT TAG ACT- 3' | (Mutant site) |
| | AEGR: 5'- TCT ACG CAC | AATATTTT |
| | TAC AGG TTA AG- 3' | |

Individually, both AEG1 and ACP1 were found to be robustly associated with cancer occurrence (see dataset 1).

AEG1 and ACP1 genotypes were combined to create a five AEG1/ACP1 risk alleles, rating subjects from low-to-high cancer risk (see dataset 2).

The AEG1/ACP1 risk alleles were found to predict the occurrence of melanoma (X2=76.611 p<0.00000), prostate cancer (X2=22.146 p<0.00000), and breast cancer (X2=10.687 p<0.001) (see dataset 3).

The aeg1/ACP1 risk alleles were found to predict the occurrence of multiple tumor sites among cancer patients (see dataset 4).

The aeg1/ACP1 risk alleles were found to predict the occurrence of multiple tumor sites among cancer patients (see dataset 5).

Dataset 1 SEPARATE EFFECTS OF ACP1
& AEG1 ON CANCER SUSCEPTIBILITY.

| | | | |
|---|---|---|---|
| Dependent Variable | CONTCA | CONTROLS = 0, | CANCER = 1 |
| By levels of | ACP1 | ACP1 | |

| Value | Label | Mean | Std Dev | Sum of Sq | Cases |
|---|---|---|---|---|---|
| 1.00 | AA/AB | .6020 | .4899 | 140.8776 | 588 |
| 2.00 | BB | .4857 | .5002 | 165.6139 | 663 |
| 3.00 | C+ | .3870 | .4880 | 61.9157 | 261 |

| | | | | |
|---|---|---|---|---|
| Within Groups Total | .5139 | .4941 | 368.4071 | 1512 |

| | Sum of | | Mean | | |
|---|---|---|---|---|---|
| Source | Squares | d.f. | Square | F | Sig. |
| Between Groups | 9.3012 | 2 | 4.6506 | 19.0489 | .0000 |
| Linearity | 9.2742 | 1 | 9.2742 | 37.9872 | .0000 |

| | |
|---|---|
| R = −.1567 | R Squared = .0246 |

| | | | |
|---|---|---|---|
| Dependent Variable | CONTCA | CONTROLS = 0, | CANCER = 1 |
| By levels of | AEG1FIR | | |

| Value | Label | Mean | Std dev | Sum of Sq | Cases |
|---|---|---|---|---|---|
| 11 | | .3764 | .4646 | 208.9045 | 890 |
| 12 | | .5731 | .4961 | 41.8263 | 171 |
| 22 | | .9286 | .2673 | .9286 | 14 |

| | | | | |
|---|---|---|---|---|
| Within Groups Total | .4149 | .4845 | 251.6692 | 1075 |

| | Sum of | | Mean | | |
|---|---|---|---|---|---|
| Source | Squares | d.f. | Square | F | Sig. |
| Between Groups | 9.2925 | 2 | 4.6463 | 19.7910 | .0000 |
| Linearity | 6.3792 | 1 | 6.3793 | 27.1729 | .0000 |

| | |
|---|---|
| R = .1562 | R Squared = .0244 |

| | ALL CA | BREAST | MELANOMA | PROSTATE |
|---|---|---|---|---|
| ACP1 | −.1567 | −.2819 | −.1886 | −.1238 |
| | (1512) | (355) | (559) | (328) |
| | p = .000 | p = .000 | p = .000 | p = .025 |
| AEG1 | .1563 | .0542 | .2694 | .1293 |
| | (1075) | (345) | (429) | (276) |
| | p = .000 | p = .315 | p = .000 | p = .032 |

DATASET 2 CONSTRUCTION OF THE GENE-GENE MODEL
AEG1 genotypes: 11 = CC; 12 = CT and 22 = TT

| | AEG1 | | | | | |
|---|---|---|---|---|---|---|
| ACP1 | 11 | 12 | 22 | AEG1/ACP1 CODING | | |
| AA/AB | .47 | .66 | 1.00 | 2 | 4 | 5 |
| | (321) | (68) | (9) | | | |
| BB | .35 | .48 | 1.00 | 1 | 2 | 5 |
| | (376) | (75) | (4) | | | |
| C+ | .30 | .59 | .00 | 1 | 3 | 1 |
| | (168) | (27) | (0) | | | |

| | | | |
|---|---|---|---|
| | SAMPLE | CONTROLS = 0, | CANCER = 1 |
| by | ACP1 | ACP1 | |
| | AEG1 | AEG1 | |

HIERARCHICAL sums of squares
Covariates entered FIRST

| | Sum of | | Mean | | Sig |
|---|---|---|---|---|---|
| Source of Variation | Squares | DF | Square | F | of F |
| Main Effects | 14.170 | 4 | 3.542 | 15.299 | .00 |
| ACP13 | 5.779 | 2 | 2.889 | 12.474 | .000 |
| AEG1GN | 8.291 | 2 | 4.195 | 18.112 | .000 |
| 2-Way Interactions | .508 | 3 | .169 | .731 | .533 |
| ACP13 AEG1GN | .508 | 3 | .169 | .731 | .533 |
| Explained | 14.678 | 7 | 2.097 | 9.052 | .000 |

DATASET 3. ASSOCIATION BETWEEN AEG1/ACP1 RISK ALLELES AND CANCER.

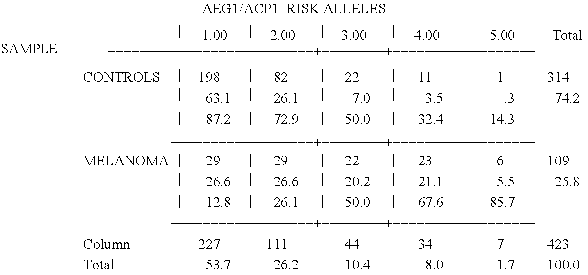

| Chi-Square | Value | DF | Significance |
|---|---|---|---|
| Pearson | 77.87240 | 4 | .00000 |
| Likelihood Ratio | 72.21857 | 4 | .00000 |
| Mantel-Haenssel test for linear association | 79.61126 | 1 | .00000 |

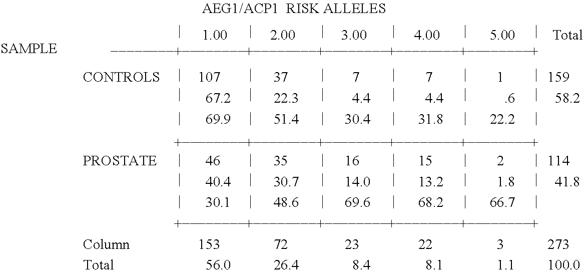

| Chi-Square | Value | DF | Significance |
|---|---|---|---|
| Pearson | 24.38495 | 4 | .00007 |
| Likelihood Ratio | 24.54770 | 4 | .00006 |
| Mantel-Haenssel test for linear association | 22.14618 | 1 | .00000 |

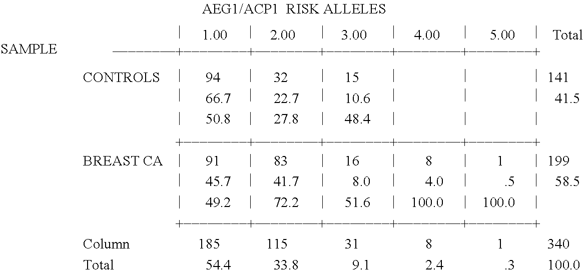

| Chi-Square | Value | DF | Significance |
|---|---|---|---|
| Pearson | 22.45771 | 4 | .00016 |
| Likelihood Ratio | 26.03899 | 4 | .00003 |
| Mantel-Haenssel test for linear association | 10.69726 | 1 | .00107 |

DATASET 4. ASSOCIATION BETWEEN AEG1/ACP1 RISK ALLELES AND MULTIPLE TUMORS.

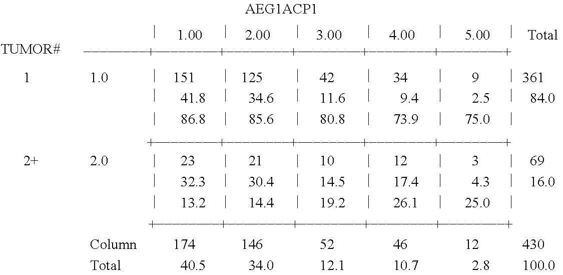

| Chi-Square | Value | DF | Significance |
|---|---|---|---|
| Pearson | 5.88049 | 4 | .20825 |
| Likelihood Ratio | 5.29977 | 4 | .24868 |
| Mantel-Haenssel test for linear association | 5.18223 | 1 | .02282 |

DATASET 5. ASSOCIATION BETWEEN AEG1/ACPI RISK ALLELES AND AGGRESSIVENESS
OF PROSTATE CANCER.

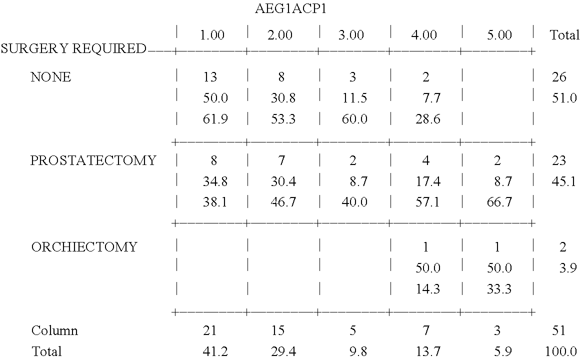

| Chi-Square | Value | DF | Significance |
|---|---|---|---|
| Pearson | 13.74945 | 8 | .08853 |
| Liklihood Ratio | 12.05298 | 8 | .14886 |
| Manel-Haenssel test for linear association | 7.64034 | 1 | .00571 |

### CITATIONS

Byoung Kwon Yoo, Luni Emdad, Seok-Geun Lee, Zao-zhong Su, Prasanna Santhekadur, Dong Chen, Gredler R, Fisher P B, Devanand S. Astrocyte Elevated Gene-1 (AEG-1): a multifunctional regulator of normal and abnormal physiology. Pharmacol Ther. 2011 130(1): 1-8.
Vogel W, Lammers R, Huang J, Ullrich A. Activation of a phosphotyrosine phosphatase by tyrosine phosphorylation. Science 1993; 259: 1611-1614.
Wo Y Y, McCormack A L, Shabanowitz J, Hunt D F, Davis J P, Mitchell G L et al. Sequencing, cloning, and expression of human red cell-type acid phosphatase, a cytoplasmic phosphotyrosyl protein phosphatase. J Biol Chem 1992; 267:10856-10865.
Malentacchi F, Marzocchini R, Gelmini S, Orlando C, Serio M, Ramponi G, Raugei G. Up-regulated expression of low molecular weight protein tyrosine phosphatases in different human cancers. Biochem Biophys Res Commun 334: 875-883, 2005.50.
Alho I, Costa L, Bicho M, Coelho C. The role of low-molecular-weight protein tyrosine phosphatase (LMW-PTP ACP1) in oncogenesis. Tumour Biol. 2013 August; 34(4):1979-89.

1. A method for genotyping polymorphisms of the ACP1 gene (rs11553742 and rs79716074), thereby creating an ACP1 haplotype that provides an accurate and reliable estimate of risk for a condition selected from the group consisting of breast cancer, malignant melanoma, and prostate cancer.

2-3. (canceled)

4. A method for genotyping polymorphisms of the AEG1 gene (rs2438211) thereby providing an accurate and reliable estimate of risk for a condition selected from the group consisting of prostate cancer and malignant melanoma.

5. (canceled)

6. A method for genotyping polymorphisms of the AEG1 gene (rs2438211) and ACP1 haplotype (rs11553742 and rs79716074), thereby creating a gene-gene combination that provides an accurate and reliable estimate of risk for a condition selected from the group consisting of prostate cancer, breast cancer, and melanoma.

7-8. (canceled)

9. A method for genotyping polymorphisms of the AEG1 gene (rs2438211) and ACP1 haplotype (rs11553742 and rs79716074), thereby creating a gene-gene combination that provides an accurate and reliable estimate among cancer patients for risk of developing a condition selected from the group consisting of multiple tumors and more aggressive forms of prostate cancer.

10. (canceled)

11. A method for modifying the biological substrates of ACP1 or AEG1 or their combined effect, thereby having the prophylactic effect selected from the group consisting of reducing cancer risk, reducing the risk of developing multiple tumor sites, and reducing the risk of more aggressive cancer progression.

12-13. (canceled)
